(19) United States
(12) Patent Application Publication
(10) Pub. No.: US 2025/0387539 A1
(43) Pub. Date: Dec. 25, 2025

(54) MENISCUS REGENERATION MATERIAL

(71) Applicants: HIROSHIMA UNIVERSITY, Hiroshima (JP); SANYO CHEMICAL INDUSTRIES, LTD., Kyoto (JP)

(72) Inventors: Nobuo ADACHI, Hiroshima (JP); Naosuke KAMEI, Hiroshima (JP); Masakazu ISHIKAWA, Hiroshima (JP); Tomoyuki NAKASA, Hiroshima (JP); Shingo KAWABATA, Kyoto (JP); Tsubasa YAMANAKA, Kyoto (JP)

(73) Assignees: HIROSHIMA UNIVERSITY, Hiroshima (JP); SANYO CHEMICAL INDUSTRIES, LTD., Kyoto (JP)

(21) Appl. No.: 19/317,507

(22) Filed: Sep. 3, 2025

Related U.S. Application Data
(63) Continuation of application No. 17/282,577, filed on Apr. 2, 2021, now Pat. No. 12,427,228.

(30) Foreign Application Priority Data
Oct. 5, 2018 (JP) 2018-190168

(51) Int. Cl. A61L 27/36; A61K 35/32; A61K 38/16; A61L 27/22; A61P 19/02; A61P 19/04; C07K 14/435; C07K 14/78

(52) U.S. Cl. CPC A61L 27/3654; A61K 35/32; A61K 38/16; A61L 27/227; A61L 27/3612; A61P 19/02; A61P 19/04; C07K 14/43586; C07K 14/78; A61L 2300/252; A61L 2300/412; A61L 2430/06

(57) ABSTRACT

The present invention aims to provide a meniscus regeneration material having high meniscus regeneration ability. The present invention provides a meniscus regeneration material including a protein (A), wherein the protein (A) contains at least one of a polypeptide chain (Y) and a polypeptide chain (Y′); a total number of the polypeptide chain (Y) and the polypeptide chain (Y′) in the protein (A) is 1 to 100; the polypeptide chain (Y) includes 2 to 200 tandem repeats of at least one amino acid sequence (X) selected from the group consisting of an amino acid sequence VPGVG (1) set forth in SEQ ID No: 1, an amino acid sequence GVGVP (4) set forth in SEQ ID No: 4, an amino acid sequence GPP, an amino acid sequence GAP, and an amino acid sequence GAHGPAGPK (3) set forth in SEQ ID No: 3; the polypeptide chain (Y′) includes the polypeptide chain (Y) in which 5% or less of amino acid residues are replaced by at least one of a lysine residue and an arginine residue, and a total number of the lysine residue and the arginine residue is 1 to 100; the protein (A) has a total percentage of β-turns and random coils of 60 to 85% as determined by circular dichroism spectroscopy; and when the amino acid sequence (X) in which 60% or less of amino acid residues are replaced by at least one of a lysine residue and an arginine residue is denoted as an amino acid sequence (X′), a ratio of a total number of amino acid residues in the amino acid sequence (X) and the amino acid sequence (X′) in the protein (A) to the number of all amino acid residues in the protein (A) is 50 to 70%.

### CROSS REFERENCE TO RELATED APPLICATIONS

The present application is a continuation-in-part of U.S. patent application Ser. No. 17/282,577, filed on Apr. 2, 2021, which is a National Stage Entry of PCT/JP2019/037,589, filed on Sep. 25, 2019, of each of which is incorporated by reference herein in its entirety.

### REFERENCE TO AN ELECTRONIC SEQUENCE LISTING

The contents of the electronic sequence listing (AttachF_SeqListing.txt; Size: 87,349 KB; and Date of Creation: Sep. 2, 2025) is herein incorporated by reference in its entirety.

### TECHNICAL FIELD

The present invention relates to meniscus regeneration materials.

### BACKGROUND ART

Osteoarthritis (OA) is the most common type of “arthritis” among elderly people. According to 2011 statistics, 25,300,000 people suffer from OA (Non-Patent Literature 1), and about 8,000,000 of them have pain or other symptoms. Knee OA is classified into primary OA and secondary OA. Primary OA is caused by factors related to aging, whereas secondary OA is caused by another disease, such as traumatic cartilage defect, meniscus damage, or anterior cruciate ligament damage. In particular, meniscus damage is considered as a trigger of knee OA, attracting worldwide attention.

Knee OA therapies so far focus on the regeneration of defective parts of joint cartilage. Various therapeutic methods have been developed and implemented (Patent Literature 1). However, when joint cartilage alone is repaired without repairing meniscus, good conditions are very difficult to maintain. Thus, a combination of a meniscus repair/regeneration therapy and alignment correction is essential for an ultimate radical cure of knee OA.

Meniscus is a very important tissue in knee joints not only for impact absorption and stabilization of the joints but also for smooth movement of the joints. The meniscus has extremely low repairability because it histologically consists of fibrocartilage in which blood vessels are scarce as in joint cartilage. Since the meniscus bears a high mechanical load, once damaged, the meniscal tissue is very difficult to repair. The most common treatment for severe symptoms (e.g., scraping feeling and pain) is arthroscopic partial meniscectomy, a symptomatic therapy. Repair and regeneration of damaged tissues are very difficult at present. Meanwhile, under the slogan of “Save the Meniscus,” a worldwide effort to repair meniscus by meniscus suture techniques is being made for knee OA prevention. However, 30% of suture surgery patients need a surgery again, suggesting a limitation of therapies of deformed meniscus which has low self-curing ability. To overcome the issue, advanced therapies are studied and developed. Examples include regenerative medicine involving the transplantation of autologous cultured synovium stem cells and collagen-containing graft materials for meniscus (Patent Literature 2). Yet, compared to cartilage damage, there is a very limited selection of therapies for meniscus damage. New approaches to cure meniscus damage are awaited.

### CITATION LIST

### Patent Literature

Patent Literature 1: JP 6338259 B

Patent Literature 2: JP 2012-236112 A

### Non-Patent Literature

Non-Patent Literature 1: International and National Guidelines of Osteoarthritis Treatment, Japanese Journal of Joint Diseases, 35(1): 1-9, 2016

### SUMMARY OF INVENTION

### Technical Problem

The present invention aims to provide a meniscus regeneration material having high meniscus regeneration ability.

### Solution to Problem

As a result of intensive studies to achieve the aim, the present inventors arrived at the present invention. Specifically, the present invention relates to a meniscus regeneration material including a protein (A), wherein
the protein (A) contains at least one of a polypeptide chain (Y) and a polypeptide chain (Y′);
a total number of the polypeptide chain (Y) and the polypeptide chain (Y′) in the protein (A) is 1 to 100;
the polypeptide chain (Y) includes 2 to 200 tandem repeats of at least one amino acid sequence (X) selected from the group consisting of an amino acid sequence VPGVG (1) set forth in SEQ ID No: 1, an amino acid sequence GVGVP (4) set forth in SEQ ID No: 4, an amino acid sequence GPP, an amino acid sequence GAP, and an amino acid sequence GAHGPAGPK (3) set forth in SEQ ID No: 3;
the polypeptide chain (Y′) includes the polypeptide chain (Y) in which 5% or less of amino acid residues are replaced by at least one of a lysine residue and an arginine residue, and a total number of the lysine residue and the arginine residue is 1 to 100;
the protein (A) has a total percentage of B-turns and random coils of 60 to 85% as determined by circular dichroism spectroscopy; and
when the amino acid sequence (X) in which 60% or less of amino acid residues are replaced by at least one of a lysine residue and an arginine residue is denoted as an amino acid sequence (X′), a ratio of a total number of amino acid residues in the amino acid sequence (X) and the amino acid sequence (X′) in the protein (A) to the number of all amino acid residues in the protein (A) is 50 to 70%.

### Advantageous Effects of Invention

The meniscus regeneration material of the present invention has excellent meniscus regeneration ability and enables normal meniscus regeneration.

### DESCRIPTION OF EMBODIMENTS

The meniscus regeneration material of the present invention includes a protein (A). The protein (A) contains at least one of a polypeptide chain (Y) and a polypeptide chain (Y′). The total number of the polypeptide chain (Y) and the polypeptide chain (Y′) in the protein (A) is 1 to 100. The polypeptide chain (Y) includes 2 to 200 tandem repeats of at least one amino acid sequence (X) selected from the group consisting of an amino acid sequence VPGVG (1) set forth in SEQ ID No: 1, an amino acid sequence GVGVP (4) set forth in SEQ ID No: 4, an amino acid sequence GPP, an amino acid sequence GAP, and an amino acid sequence GAHGPAGPK (3) set forth in SEQ ID No: 3. The polypeptide chain (Y′) includes the polypeptide chain (Y) in which 5% or less of amino acid residues are replaced by at least one of a lysine residue and an arginine residue, and a total number of the lysine residue and the arginine residue is 1 to 100. The protein (A) has a total percentage of β-turns and random coils of 60 to 85% as determined by circular dichroism spectroscopy. When the amino acid sequence (X) in which 60% or less of amino acid residues are replaced by at least one of a lysine residue and an arginine residue is denoted as an amino acid sequence (X′), a ratio of a total number of amino acid residues in the amino acid sequence (X) and the amino acid sequence (X′) in the protein (A) to the number of all amino acid residues in the protein (A) is 50 to 70%.

The meniscus regeneration material of the present invention includes a protein (A). The protein (A) contains at least one of a polypeptide chain (Y) and a polypeptide chain (Y′). Such a material can maintain an appropriate wet state of a gel described in detail later.

The polypeptide chain (Y) may include one type or two or more types of the amino acid sequence (X).

To maintain an appropriate wet state of a gel described in detail later and to enhance the meniscus regeneration ability, preferred examples of the amino acid sequence (X) include a VPGVG sequence (1) and a GVGVP sequence (4).

Specific examples of the polypeptide chain (Y) include a (VPGVG)_{b} sequence, a (GVGVP)_{c} sequence, and a (GAHGPAGPK)_{d} sequence. The symbols b, c, and d each represent the number of tandem repeats of the amino acid sequence (X), and the number is an integer of 2 to 200.

When the protein (A) contains a plurality of the polypeptide chains (Y) in one molecule, the polypeptide chains (Y) may be the same as or different from each other. The protein (A) may contain one sequence selected from the group consisting of a (VPGVG)_{b} sequence, a (GVGVP)_{c} sequence, and a (GAHGPAGPK)_{d} sequence, or two or more of the sequences.

When the protein (A) contains a plurality of the polypeptide chains (Y), the polypeptide chains (Y) have the same number or different numbers of tandem repeats of the amino acid sequence (X). Specifically, the protein (A) may contain a plurality of the polypeptide chains (Y) having the same or different number (b, c, or d) of tandem repeats of the amino acid sequence (X).

To maintain an appropriate wet state of a gel described in detail later, preferred examples of the polypeptide chain (Y) include a (VPGVG)_{b} sequence and a (GVGVP)_{c} sequence.

The polypeptide chain (Y) includes 2 to 200 tandem repeats of the amino acid sequence (X) (i.e. each of b, c, and d represents an integer of 2 to 200). To maintain an appropriate wet state of a gel, the number of the tandem repeats of the amino acid sequence (X) is preferably 2 to 100 (i.e. each of b, c, and d represents an integer of 2 to 100), still more preferably 2 to 50 (i.e. each of b, c, and d represents an integer of 2 to 50), particularly preferably 2 to 40 (i.e. each of b, c, and d represents an integer of 2 to 40).

The polypeptide chain (Y′) includes the polypeptide chain (Y) in which 5% or less of amino acid residues are replaced by at least one of a lysine residue and an arginine residue, and the total number of the replacing lysine residue and the replacing arginine residue is 1 to 100.

Identification of the polypeptide chain (Y′) is made based on whether the polypeptide chain Y is obtained when all of lysine (K) residues and arginine (R) residues in the sequence of the protein (A) are replaced by other amino acid residues (glycine (G), alanine (A), valine (V), proline (P) or histidine (H)).

To maintain an appropriate wet state of a gel described in detail later, the percentage of the at least one of the replacing lysine residue and the replacing arginine residue in the polypeptide chain (Y′) is preferably 0.06 to 58, more preferably 0.5 to 5%, particularly preferably 1 to 5%.

The polypeptide chain (Y′) may contain an amino acid sequence (X′) that is the amino acid sequence (X) in which 60% or less of amino acid residues are replaced by at least one of a lysine residue and an arginine residue.

The polypeptide chain (Y′) may include one type or two or more types of the amino acid sequence (X) and/or one type or two or more types of the amino acid sequence (X′).

Specific examples of the amino acid sequence (X′) include an amino acid sequence GKGVP (7) set forth in SEQ ID No: 7, an amino acid sequence GKGKP (8) set forth in SEQ ID No: 8, an amino acid sequence GKGRP (9) set forth in SEQ ID No: 9, and an amino acid sequence GRGRP (10) set forth in SEQ ID No: 10.

To maintain a meniscus-defected face in an appropriate wet state, the amino acid sequence (X′) preferably includes at least one sequence selected from the group consisting of the GKGVP sequence (7), the GKGKP sequence (8), and the GRGRP sequence (10), and still more preferably includes the GKGVP sequence (7) and/or the GKGKP sequence (8).

The total number of the polypeptide chain (Y) and the polypeptide chain (Y′) in one molecule of the protein (A) is 1 to 100. The total number is preferably 1 to 80, more preferably 1 to 60.

When the total number of the polypeptide chain (Y) and the polypeptide chain (Y′) in one molecule of the protein (A) is within the range, an appropriate wet state of a gel described in detail later can be maintained.

When the protein (A) contains the polypeptide chains (Y) each having a different amino acid sequence (X) and/or a different number of tandem repeats of the amino acid sequence (X), the number of the polypeptide chains (Y) refers to the total number of the polypeptide chains (Y) each counted as one. The same applies to the polypeptide chain (Y′).

In the meniscus regeneration material of the present invention, a percentage of the total number of amino acid residues in the amino acid sequence (X) and the amino acid sequence (X′) in the protein (A) in the number of all amino acid residues in the protein (A) is 50 to 70%.

The material in which the percentage is lower than 50% does not easily become a gel as described in detail later. The material in which the percentage is higher than 70% does not dissolve well in water.

To enhance the meniscus regeneration ability, the percentage is preferably 52.5 to 67.5%, more preferably 55 to 65%.

The percentage may be determined with a protein sequencer, specifically by the following measurement method.
<Determination of percentage of total number of amino acid residues in amino acid sequence (X) and amino acid sequence (X′) in the number of all amino acid residues in protein (A)>

The protein (A) is divided into fragments of about not more than 30 amino acid residues using two or more techniques which can cleave the sequence at specific amino acid residues. Next, the fragments are separated by high-performance liquid chromatography (HPLC), and then the amino acid sequences are analyzed with a protein sequencer. The complete amino acid sequence of the protein (A) is determined by peptide mapping of the amino acid sequence obtained. Then, a percentage of the total number of amino acid residues in all the amino acid sequences (X) and amino acid residues in all the amino acid sequences (X′) in the number of all amino acid residues in the protein (A) is calculated using the following equation:

$$Percentage ( \% ) of total number of amino acid residues in all amino acid sequences ( X ) in protein ( A ) and amino acid residues in all amino acid sequences ( {X}^{′} ) in protein ( A ) = 100 \times [ \{ number of amino acid sequences ( X ) \} \times \{ number of amino acid residues in amino acid sequence ( X ) \} + \{ number of amino acid sequences ( {X}^{′} ) \} \times \{ number of amino acid residues in amino acid sequence ( {X}^{′} ) \} ] / \{ number of all amino acid residues in protein ( A ) \}$$

To control the total percentage of the β-turns and the random coils in the protein (A), which are described later, within a prescribed range, to maintain an appropriate wet state of a gel, and to enhance the meniscus regeneration ability, the protein (A) preferably contains a polypeptide chain (S) including 2 to 50 tandem repeats of a GAGAGS sequence (2).

To maintain an appropriate wet state of a gel, the number of the tandem repeats of the GAGAGS sequence (2) in the polypeptide chain (S) is preferably 2 to 40, more preferably 2 to 30, particularly preferably 2 to 10.

To enhance the meniscus regeneration ability, the percentage of the number of the amino acid residues in all the GAGAGS sequences (2) in the protein (A) in the number of all amino acid residues in the protein (A) [100×{number of GAGAGS sequences (2) in protein (A)×6}/{number of all amino acid residues in protein (A)}] is preferably 5 to 50%, more preferably 10 to 47.5%, particularly preferably 20 to 45%.

The percentage of the number of the amino acid residues in all the GAGAGS sequences (2) in the number of all amino acid residues in the protein (A) can be determined with a protein sequencer. Specifically, the percentage is determined by the following measurement method.
<Percentage of the number of amino acid residues in all GAGAGS sequences (2) in protein (A) in the number of all amino acid residues in protein (A)>

The protein (A) is divided into fragments of about not more than 30 amino acid residues using two or more techniques which can cleave the sequence at specific amino acid residues. Next, the fragments were separated by high-performance liquid chromatography (HPLC), and then the amino acid sequence is analyzed with a protein sequencer. The complete amino acid sequence of the protein (A) is determined by peptide mapping of the amino acid sequence obtained. Then, a percentage of the number of the amino acid residues in all the GAGAGS sequences (2) in the protein (A) in the number of all amino acid residues in the protein (A) is calculated using the following equation:

$$Percentage of number of amino acid residues in all GAGAGS sequences ( 2 ) in protein ( A ) in the number of all amino acid residues in protein ( A ) ( \% ) = [ \{ number of GAGAGS sequences ( 2 ) \times 6 \} / \{ number of all amino acid residues in protein ( A ) \} ] \times 100$$

In the case where the protein (A) contains total two or more of at least one polypeptide chain selected from the group consisting of a polypeptide chain (Y), a polypeptide chain (Y′), and a polypeptide chain (S), it may contain an intervening amino acid sequence (Z) between the polypeptide chains. The intervening amino acid sequence (Z) refers to a peptide sequence which consists of one amino acid residue or two or more amino acid residues linked to each other and is not any one of the GAGAGS sequence (2), the amino acid sequence (X), and the amino acid sequence (X′). To maintain an appropriate wet state of a gel described in detail later, the number of amino acid residues in the intervening amino acid sequence (Z) is preferably 1 to 30, more preferably 1 to 15, particularly preferably 1 to 10. Specific examples of the intervening amino acid sequence (Z) include an amino acid sequence VAAGY (11) set forth in SEQ ID No: 11, an amino acid sequence GAAGY (12) set forth in SEQ ID No: 12, and a LGP sequence.

To maintain an appropriate wet state of a gel described in detail later, the percentage of the amino acid residues in all the intervening amino acid sequence(s) (Z) in the number of all amino acid residues in the protein (A) [100×Σ{(number of amino acid residues in intervening amino acid sequence (Z))×(number of intervening amino acid sequences (Z))}/{number of all amino acid residues in protein (A)}] is preferably 0 to 25%, more preferably 0 to 22.5%, particularly preferably 0.01 to 15%.

From the standpoint of degradability in vivo, the protein (A) may contain a terminal amino acid sequence (T) at an end thereof, other than the GAGAGS sequence (2), the amino acid sequence (X), the amino acid sequence (X′), and the intervening amino acid sequence (Z). The terminal amino acid sequence (T) may be present at one end or both ends of the protein (A).

The terminal amino acid sequence (T) does not include a purification tag described later.

The protein (A) preferably has an end structure where the terminal amino acid sequence (T) is linked to the polypeptide chain (Y). The terminal amino acid sequence (T) refers to a peptide sequence which consists of one amino acid residue or two or more amino acid residues linked to each other and is not any one of the GAGAGS sequence (2), the amino acid sequence (X), and the amino acid sequence (X′). From the standpoint of degradability in vivo, the number of amino acid residues in the terminal amino acid sequence (T) is preferably 1 to 100, more preferably 1 to 50, particularly preferably 1 to 40. Specific examples of the terminal amino acid sequence (T) include an amino acid sequence MDPVVLQRRDWENPGVTOLNRLAAHPPFASDPM (13) set forth in SEQ ID No: 13.

From the standpoint of degradability in vivo, the percentage of the number of amino acid residues in the terminal amino acid sequence (T) in the number of all amino acid residues in the protein (A) is preferably 0 to 25%, more preferably 0 to 22.5%, particularly preferably 0.01 to 15%.

The protein (A) may be produced by a biotechnological approach using bacteria as described later. In this case, the protein (A) may include a protein or peptide (hereinafter referred to as “purification tag”) having, at the N terminal or C terminal, a specific amino acid sequence other than the terminal amino acid sequence (T) to facilitate the purification or detection of the expressed protein (A). An affinity purification tag is used as the purification tag. Examples of the purification tag include 6×His tag containing polyhistidine, V5 tag, Xpress tag, AU1 tag, T7 tag, VSV-G tag, DDDDK tag, S tag, CruzTag09™, CruzTag22™, CruzTag41™, Glu-Glu tag, Ha.11 tag, and KT3 tag.

Exemplary combinations of a purification tag (i) and a ligand (ii) capable of recognizing and binding to the tag are listed below.
(i-1) glutathione-S-transferase (GTS) and (ii-1) glutathione
(i-2) maltose-binding protein (MBP) and (ii-2) amylose
(i-3) HQ tag and (ii-3) nickel
(i-4) Myc tag and (ii-4) anti-Myc antibody
(i-5) HA tag and (ii-5) anti-HA antibody
(i-6) FLAG tag and (ii-6) anti-FLAG antibody
i-7) 6×His tag and (ii-7) nickel or cobalt

The purification tag sequence may be added by incorporating a nucleic acid encoding the purification tag into the 5′ or 3′ end of a nucleic acid encoding the protein (A) in an expression vector or using a commercial vector designed to add the purification tag, for example.

From the standpoint of degradability in vivo, the percentage of a sum of the number of amino acid residues in all the intervening amino acid sequence(s) (Z) in the protein (A), the number of amino acid residues in all the terminal amino acid sequence(s) (T) in the protein (A), and the number of amino acid residues in the purification tag in the number of all amino acid residues in the protein (A) is preferably 0 to 25%, more preferably 0 to 22.5%, particularly preferably 0.01 to 15%.

To maintain a defected face in an appropriate wet state, in the case where the protein (A) contains the polypeptide chain (S) in addition to the at least one of the polypeptide chain (Y) and the polypeptide chain (Y′), preferably the polypeptide chain (Y) or the polypeptide chain (Y′) and the polypeptide chain (S) are alternately linked by a chemical bond.

The ratio of the number of the GAGAGS sequence (2) to the total number of the amino acid sequence (X) and the amino acid sequence (X′) (number of GAGAGS sequence (2): total number of amino acid sequence (X) and amino acid sequence (X′)) is preferably 1:1.5 to 1:20, more preferably 1:1.5 to 1:6, particularly preferably 1:2 to 1:5 to give a moderate total percentage of the β-turns and the random coils in the protein (A).

Some preferred examples of the protein (A) are listed below.

(A1): a protein in which the amino acid sequence (X) is the GVGVP sequence (4);
(A11): a protein containing a polypeptide chain (Y′1) having an amino acid sequence which includes 2 to 200 tandem repeats of the GVGVP sequence (4) and in which one amino acid residue is replaced by a lysine (K) residue;
(A11-1): a protein containing the polypeptide chain (Y′1) and a polypeptide chain (S1) including 2 to 200 tandem repeats of the GAGAGS sequence (2);
(A11-2): a protein containing an amino acid sequence (GVGVP)₄GKGVP (GVGVP)₃ (6) set forth in SEQ ID No: 6 (Y′11) in which one amino acid residue in a polypeptide chain (Y11) having an amino acid sequence (GVGVP)₈ (14) set forth in SEQ ID No: 14 including 8 tandem repeats of a GVGVP sequence (4) is replaced by a lysine (K) residue and the polypeptide chain (S1) including 2 to 200 tandem repeats of the GAGAGS sequence (2); and
(A11-2-1): a protein containing a polypeptide chain (S1-1) having an amino acid sequence (GAGAGS)₄ (5) set forth in SEQ ID No: 5 including 4 tandem repeats of the GAGAGS sequence (2) and a (GVGVP)₄GKGVP (GVGVP)₃ sequence (6).

Specific examples include the following proteins:
(i) a protein (SELP8K) having an amino acid sequence (16) set forth in SEQ ID No: 16 having a molecular mass of about 80 kDa. The sequence (16) includes 12 (GAGAGS)₄ sequences (5) and 13 (GVGVP)₄GKGVP (GVGVP)₃ sequences (6), which are alternately linked by a chemical bond, and an amino acid sequence (GAGAGS)₂ (15) set forth in SEQ ID No: 15 linked by a chemical bond to the linked sequences;
(ii) a protein (SELP0K) having an amino acid sequence (17) set forth in SEQ ID No: 17 having a molecular mass of about 82 kDa. The sequence (17) has a structure in which 17 (GAGAGS)₂ sequences (15) and 17 (GVGVP)₄GKGVP (GVGVP)₃ sequences (6) are alternately linked by a chemical bond;
(iii) a protein (SELP8K4) having an amino acid sequence (29) set forth in SEQ ID No: 29 having a molecular mass of about 30 kDa and including 4 (GAGAGS)₄ sequences (5) and 4 (GVGVP)₄GKGVP (GVGVP) 3 sequences (6), which are alternately linked by a chemical bond;
(A11-3): a protein containing a polypeptide chain (Y′12) having an amino acid sequence (GVGVP)₆GKGVP (GVGVP)₅ (18) set forth in SEQ ID No: 18 in which one amino acid residue in a polypeptide chain including 12 tandem repeats of the GVGVP sequence (4) is replaced by lysine (K) and the polypeptide chain (S1) including 2 to 200 tandem repeats of the GAGAGS sequence (2);
(A11-3-1): a protein having an amino acid sequence (GAGAGS)₄ (19) set forth in SEQ ID No: 19 including 4 tandem repeats of the GAGAGS sequence (2) and a (GVGVP)₆GKGVP (GVGVP)₅ sequence (18);
(i) a protein having an amino acid sequence (20) set forth in SEQ ID No: 20 having a molecular mass of about 105 kDa. The sequence (20) includes 12 (GAGAGS)₄ sequences (19) and 13 (GVGVP)₆GKGVP (GVGVP)₅ sequences (18), which are alternately linked by a chemical bond, and the (GAGAGS)₂ sequence (15) linked by a chemical bond to the linked sequences;
(A2): a protein in which the amino acid sequence (X) is the VPGVG sequence (1);
(A21): a protein containing a polypeptide chain (Y2) including 2 to 200 tandem repeats of the VPGVG sequence (1) and the GAGAGS sequence (2);
(i) a protein (ELP1.1) having an amino acid sequence (26) set forth in SEQ ID No: 26 having a molecular mass of about 200 kDa. The sequence (26) includes 40 GAGAGS sequences (2), 40 amino acid sequences (VPGVG)₄ (24) set forth in SEQ ID No: 24, and 40 amino acid sequences (VPGVG)₈ (25) set forth in SEQ ID No: 25, which are arranged such that 40 blocks each consisting of one (VPGVG)₄ sequence (24), one GAGAGS sequence (2), and one (VPGVG)₈ sequence (25) bound in said order are linked by a chemical bond; and
(A3): a protein containing the polypeptide chain (Y1) including 2 to 200 tandem repeats of the GVGVP sequence (4) and the polypeptide chain (S1) including 2 to 200 tandem repeats of the GAGAGS sequence (2).

Specific examples also include the following protein:
(i) a protein (SELP6.1) having an amino acid sequence (23) set forth in SEQ ID No: 23 having a molecular mass of about 110 kDa. The sequence (23) includes 5 amino acid sequences (GAGAGS)₈ (21) set forth in SEQ ID No: 21 and 5 amino acid sequences (GVGVP)₄₀ (22) set forth in SEQ ID No: 22, which are alternately linked by a chemical bond.

Preferred among these are the protein (SELP8K) having the sequence (16), the protein (SELP0K) having the sequence (17), the protein having the sequence (20), the protein (SELP6.1) having the sequence (23), the protein (ELP1.1) having the sequence (26), or the protein (SELP8K4) having the sequence (29).

Examples of the protein (A) also include proteins each having an amino acid sequence which is not less than 70% identical to the amino acid sequence of the protein (SELP8K) having the sequence (16), the protein (SELP0K) having the sequence (17), the protein having the sequence (20), the protein (SELP6.1) having the sequence (23), the protein (ELP1.1) having the sequence (26), or the protein (SELP8K4) having the sequence (29).

The percentage of identicalness is preferably not less than 80%, more preferably not less than 90%.

The protein (A) in the present invention has a total percentage of β-turns and random coils of 60 to 85% as determined by circular dichroism spectroscopy. Proteins having an identical sequence may have different total percentages of β-turns and random coils depending on, for example, the methods of producing the proteins, the methods of purifying the proteins, the pH of solvents for dissolving the proteins, or the polarities of the solvents.

The protein (A) having the total percentage of β-turns and random coils of lower than 60% does not dissolve well in water. The protein having the total percentage of higher than 85% does not easily becomes a gel as described in detail later.

To maintain a defected face in an appropriate wet state and promote meniscus regeneration, the total percentage of β-turns and random coils in the protein (A) is preferably 65 to 80%, more preferably 70 to 75%.

The percentage can be controlled by any method and can be increased or decreased by the following methods.

Examples of the method to increase the percentage include a dilution refolding method (high dilution method) in which a single protein (A) is diluted with excess buffer and then refolded.

Examples of the method to reduce the percentage include modifying the protein (A) with a denaturant, heat, or the like.

The total percentage of β-turns and random coils in the protein (A) is determined by the following measurement.
<Measurement of total percentage of β-turns and random coils in protein (A)>

The protein is dissolved in deionized water (4° C.) to a concentration of 0.3 mg/mL to prepare an aqueous solution of the protein. The aqueous solution of the protein is measured with a circular dichroism spectrometer (JASCO Corporation, J-820) at a measurement temperature of 4° C. The percentage of β-turns and the percentage of random coils are calculated using a secondary structure analysis program (JWSSE model, JASCO Corporation), and the sum of the percentages is determined as a total percentage of β-turns and random coils.

From the standpoint of degradability in vivo, the protein (A) has a molecular mass as determined by SDS-PAGE (SDS polyacrylamide gel electrophoresis) of preferably 15 to 200 kDa, more preferably 30 to 150 kDa, particularly preferably 70 to 120 kDa.

To enhance the meniscus regeneration ability, the protein (A) in the present invention has a hydrophobicity of preferably 0.2 to 1.2, more preferably 0.4 to 1.0, particularly preferably 0.42 to 0.80.

The hydrophobicity of the protein (A) refers to a degree of hydrophobicity of a protein (A) molecule. The hydrophobicity can be calculated by inserting the number (M_{α}) of each amino acid residue in one molecule of the protein (A), the hydrophobicity (N_{α}) of each amino acid residue, and the total number (M_{T}) of amino acid residues in one molecule of the protein (A) in the equation below. The hydrophobicities of the amino acid residues are below-mentioned values described in a non-patent literature (Albert L. Lehninger, David L. Nelson, Lehninger, Principles of Biochemistry, Vol. 1, Hirokawa Shoten Co., September 2010, pp. 346-347).

$$Hydrophobicity = \sum ( {M}_{α} \times {N}_{α} ) / ( {M}_{T} )$$

M_{α}: number of each amino acid residue in one molecule of artificial protein (A)
N_{α}: hydrophobicity of the amino acid residue
M_{T}: total number of amino acid residues in one molecule of artificial protein (A)
A (alanine): 1.8
R (arginine): −4.5
N (asparagine): −3.5
D (aspartic acid): −3.5
C (cysteine): 2.5
Q (glutamine): −3.5
E (glutamic acid): −3.5
G (glycine): −0.4
H (histidine): −3.2
I (isoleucine): 4.5
L (leucine): 3.8
K (lysine): −3.9
M (methionine): 1.9
F (phenylalanine): 2.8
P (proline): −1.6
S (serine): −0.8
T (threonine): −0.7
W (tryptophan): −0.9
Y (tyrosine): −1.3
V (valine): 4.2

For example, the hydrophobicity of an artificial protein (A) having the (GVGVP)₄GKGVP (GVGVP)₃ sequence (6) is calculated as follows:

$$Hydrophobicity of artificial protein ( A ) = \{ 16 ( number of G ) \times ( - 0.4 ) + 15 ( number of V ) \times 4.2 + 8 ( number of P ) \times ( - 1.6 ) + 1 ( number of K ) \times ( - 3.9 ) \} / 40 ( total number of amino acid residues ) = 1. .$$

The meniscus regeneration material of the present invention includes the protein (A) having the amino acid sequence described above. Such a material is decomposed by an enzyme in vivo and thus has excellent biodegradability.

The protein (A) in the present invention can be obtained by extraction from natural products or by organic synthesis (e.g., enzymatic method, solid phase synthesis, and liquid phase synthesis) or gene recombination. Examples of the organic synthesis include the method described in “Seikagaku Jikken Koza 1 (Biochemistry Experimental Course 1), Chemistry of protein IV (edited by The Japanese Biochemical Society, published by Tokyo Kagakudojin on Jul. 1, 1981)” or “Zoku Seikagaku Jikken Koza 2) (Sequel to Biochemistry Experimental Course 2, Chemistry of protein (Vol. 2) (edited by The Japanese Biochemical Society, published by Tokyo Kagakudojin on May 20, 1987).” Examples of the gene recombination include the method described in JP 3338441 B. The protein (A) can be produced by extraction from natural products, organic synthesis, and gene recombination. Yet, the gene recombination is preferred for easy alteration of amino acid sequences and mass productivity at low cost.

To enhance the meniscus regeneration ability, the meniscus regeneration material of the present invention preferably includes a meniscus tissue fragment (B).

The meniscus tissue fragment (B) may be obtained by collecting a piece of the meniscus (it may be the meniscus of the patient or the meniscus of a person other than the patient), and finely grinding the meniscus piece.

The meniscus piece can be finely ground with a scalpel, for example.

To enhance the meniscus regeneration ability, the number average volume per fragment of the meniscus tissue fragment (B) is preferably 0.001 to 1,000 mm³, more preferably 0.008 to 970 mm³, particularly preferably 1 to 500 mm³.

In addition to the protein (A) and the meniscus tissue fragment (B), the meniscus regeneration material of the present invention may further include water, an inorganic salt, and/or a phosphoric acid (salt).

Specific examples of the inorganic salt include sodium chloride, potassium chloride, calcium chloride, magnesium chloride, sodium sulfate, potassium sulfate, calcium sulfate, magnesium sulfate, sodium hydrogen carbonate, potassium hydrogen carbonate, calcium hydrogen carbonate, and magnesium hydrogen carbonate. Herein, the inorganic salt does not include phosphoric acids (salts).

To enhance the meniscus regeneration ability, the inorganic salt content (% by weight) of the meniscus regeneration material based on the weight of the meniscus regeneration material is preferably 0 to 3% by weight, more preferably 0 to 1% by weight, particularly preferably 0.001 to 0.5% by weight.

The phosphoric acid (salt) refers to phosphoric acid and/or a phosphate.

Examples of the phosphate include alkali metal salts and alkaline earth metal salts of phosphoric acid. Specific examples include sodium salts, potassium salts, calcium salts, and magnesium salts of phosphoric acid.

To enhance the meniscus regeneration ability, the phosphoric acid (salt) content of the meniscus regeneration material based on the weight of the meniscus regeneration material is preferably 0.001 to 2% by weight, more preferably 0.001 to 0.5% by weight, particularly preferably 0.001 to 0.05% by weight.

To enhance the meniscus regeneration ability, the weight percentage of the protein (A) contained in the meniscus regeneration material of the present invention in the meniscus regeneration material is preferably 5 to 25% by weight, more preferably 10 to 20% by weight.

To enhance the meniscus regeneration ability, the weight percentage of the meniscus tissue fragment (B) contained in the meniscus regeneration material of the present invention in the meniscus regeneration material is preferably 5 to 50% by weight, more preferably 10 to 40% by weight.

To further enhance the meniscus regeneration ability, a ratio of the weight of the protein (A) to the weight of the meniscus tissue fragment (B) (weight of protein (A)/weight of meniscus tissue fragment (B)) in the meniscus regeneration material of the present invention is preferably 0.30 to 0.90, more preferably 0.40 to 0.75.

The meniscus regeneration material of the present invention may be produced by adding the meniscus tissue fragment (B) to an aqueous solution of the protein (A) and mixing them with a pipette, for example. This operation is preferably performed at 4° C. to 10° C. to maintain the cell activity.

For example, the meniscus regeneration material of the present invention may be used as follows:
(1) The meniscus regeneration material of the present invention is warmed to 4° C. to 40° C. (preferably 20° C. to 40° C.) to be a gel.

The meniscus regeneration material gel is administered to a damaged part of the meniscus exposed by incision or the like, thereby promoting the regeneration of the meniscus. After the administration of the meniscus regeneration material, the incision can be sutured and sealed with a collagen membrane, the synovium of the patient, or the like.
(2) The meniscus regeneration material of the present invention is administered to a damaged part of the meniscus under an arthroscope. The meniscus regeneration material is made into a gel by the body temperature of the patient. Thus, as in the above (1), the material can promote the regeneration of the meniscus.

The methods correspond to the therapies of a damaged part of the meniscus of the present invention.

In a method of the present invention, the meniscus regeneration material is in at least one form selected from the group consisting of solid, aqueous solution, and gel. In some methods of the present invention the meniscus regeneration material is in a solid form. In some methods of the present invention the meniscus regeneration material is in the form of an aqueous solution. In some methods of the present invention the meniscus regeneration material is in the form of a gel. In a method where the meniscus regeneration material is in the solid form, the material is in at least one form selected from powder, sponge, fiber, sheet and non-woven fabric.

The meniscus regeneration material of the present invention may be used for osteoarthritis, cartilage defect, and meniscus damage (traumatic meniscus damage, etc.), for example.

The use of the meniscus regeneration material for at least one disease selected from the group consisting of osteoarthritis, cartilage defect, and meniscus damage (traumatic meniscus damage) corresponds to the use of the meniscus regeneration material of the present invention.

### EXAMPLES

The present invention will be more specifically described below with reference to examples, but the present invention is not limited to the examples.

### Production Example A1: Preparation of SELP8K (A11-2-1 (i))

### Production of SELP8K (A11-2-1 (i))

A plasmid pPT0345 encoding SELP8K was prepared by the method described in Examples in JP 4088341 B.

The plasmid was used to transform Escherichia coli cells into a strain capable of producing SELP8K. The following describes a method for producing the protein (A) having the sequence (16) using the SELP8K-producing strain.

### Culture of SELP8K-Producing Strain

A culture solution prepared by culturing the SELP8K-producing strain at 30° C. overnight was inoculated to 50 mL of LB medium in a 250 mL flask. Kanamycin was added to a final concentration of 50 mg/mL, and the culture solution was incubated with agitation (200 rpm) at 30° C. When the turbidity (OD 600) of the inoculated culture solution reached 0.8 (measured with spectrophotometer UV1700, Shimadzu Corporation), 40 mL of the culture solution was transferred to another flask pre-warmed at 42° C. and incubated at the same temperature for about two hours. The culture solution was chilled on ice, and the turbidity (OD 600) of the culture solution was measured. Escherichia coli cells were collected by centrifugation.

### Purification of SELP8K (A11-2-1 (i))

The collected Escherichia coli cells were used to purify a protein from an Escherichia coli biomass through the following 1 to 7; 1: Lysis, 2: Removal of insoluble cellular debris by centrifugation, 3: Ammonium sulfate precipitation, 4: Ultrafiltration, 5: Anion exchange chromatography, 6: Ultrafiltration, and 7: Lyophilization. Accordingly, a protein (A) in Production Example A1 (SELP8K) that is a purified protein having a molecular mass of about 80 kDa and having the sequence (16) was obtained.

### 1: Lysis

Deionized water (200 g) was added to 100 g of the collected Escherichia coli cells), followed by lysis with a high-pressure homogenizer (55 MPa). Thus, a lysate containing lysed cells was obtained. Subsequently, the pH of the lysate was adjusted to 4.0 with glacial acetic acid.

### 2: Removal of Insoluble Cellular Debris by Centrifugation

The lysate was further centrifuged (6, 300 rpm, 4° C., 30 min) to collect the supernatant.

### 3: Ammonium Sulfate Precipitation

A saturated ammonium sulfate solution was added to the collected supernatant to give an ammonium sulfate concentration of 25% by weight, followed by standing for 8 to 12 hours. Then, the precipitate was collected by centrifugation. The collected precipitate was dissolved in deionized water. To the solution was added a saturated ammonium sulfate solution to similarly give an ammonium sulfate concentration of 25% by weight, followed by standing for 8 to 12 hours. Then, the precipitate was collected by centrifugation. The collected precipitate was dissolved in deionized water to obtain a solution.

### 4: Ultrafiltration

The solution prepared in “3: Ammonium sulfate precipitation” was applied to an ultrafilter with a cut-off molecular mass of 30,000 Da (Hollow Fiber, GE Healthcare). The solution prepared in “3: Ammonium sulfate precipitation” was ultrafiltered against deionized water in an amount 10 times the volume of the solution. Thus, the protein was separated by ultrafiltration.

### 5: Anion Exchange Chromatography

The protein separated by ultrafiltration was dissolved in a 10 mM sodium acetate buffer to a concentration of 20 g/L, and applied to AKTA Prime (GE Healthcare) to which an anion exchange column (Hi PrepSP XL16/10, GE Healthcare) was connected. Then, using 500 mM of a 10 mM sodium acetate buffer as an eluate, an eluted fraction was collected.

### 6: Ultrafiltration

The solution obtained in “5: Anion exchange chromatography” was treated in the same manner as in “(4) Ultrafiltration” to separate the protein.

### 7: Lyophilization

The protein was dissolved in deionized water to give a 5 g/L solution. The solution was poured into a stainless-steel vat such that the water level would be 15 mm or lower. The vat was placed in a lyophilizer (NIHON TECHNO SERVICE CO., LTD.) to freeze the solution at −40° C. over 16 hours. The frozen product was subjected to primary drying at a vacuum of 8 Pa or less at −20° C. over 90 hours, and then to secondary drying at a vacuum of 8 Pa or less at 20° C. over 24 hours. Accordingly, a protein (A) in Production Example A1 (SELP8K) was obtained.

### Identification of SELP8K (A11-2-1 (i))

The protein (A) in Production Example A1 was identified by the following procedure.

The protein was analyzed by Western Blotting using a rabbit anti-SELP8K antibody and a rabbit anti-6×His antibody (Roland Corporation) against 6×His tag at the C terminal. Western Blotting was performed by the procedure described later. A band exhibiting reactivity with each antibody was found at an apparent molecular mass of 80 kDa.

Table 1 shows the percentages of the amino acid residues (measured values) in the protein (A) in Production Example A1 determined by an amino acid analysis using an amino acid analysis system (Prominence, Shimadzu Corporation) and the percentages of the amino acid residues (theoretical values) in SELP8K estimated from a synthetic gene sequence.

The protein (A) in Production Example A1 was confirmed as a protein (SELP8K) having the sequence (16) including 13 polypeptide chains (Y′2) having a (GVGVP)₄GKGVP (GVGVP)₃ sequence (6) and 12 polypeptide chains (S1-1) having an amino acid sequence (GAGAGS)₄ (5), where these polypeptide chains are alternately linked by a chemical bond. Here, the polypeptide chain (Y′2) is a polypeptide chain in which one of valines (V) in the polypeptide chain (Y) consisting of 8 tandem repeats of the GVGVP sequence (4) is replaced by a lysine (K) residue, and the amino acid sequence (GAGAGS)₄ (5) includes 4 tandem repeats of the GAGAGS sequence (2).

TABLE 1

| | Measured | Theoretical |
|---|---|---|
| | value | value |
| Amino | Percentage | Percentage |
| acid | (%) | (%) |
| Ala | 12.3 | 12.2 |
| Asx | 0.9 | 0.8 |
| Glx | n.d. | 0.4 |
| Phe | 0.4 | 0.1 |
| Gly | 43.7 | 41.5 |
| His | 0.4 | 0.8 |
| Ile | 0.3 | 0 |
| Lys | 1.5 | 1.5 |
| Leu | 0.3 | 0.5 |
| Met | 0.3 | 0.3 |
| Pro | 11.7 | 12.4 |
| Arg | 0.5 | 0.6 |
| Ser | 5.3 | 6.1 |
| Thr | n.d. | 0.1 |
| Val | 21.2 | 22.4 |
| Tyr | 1.1 | 0.1 |

### Western Blotting

A sample for Western Blotting (20 μL) was mixed with 10 mL of 3×SDS treatment buffer (150 mM Tris HCl (pH 6.8), 300 mM dithiothreitol, 6% dodecyl sodium sulfate (SDS), 0.3% bromophenol blue, and 30% glycerol) and heated at 95° C. for five minutes. Thus, a sample for electrophoresis was prepared. SDS-PAGE was carried out with 15 μL of the sample for electrophoresis. After electrophoresis, the gel was transferred to a polyvinylidene fluoride membrane (hereinafter also simply referred to as “membrane”), and immersed in a blocking buffer (20 mM Tris (pH 7.6), 137 mM NaCl, 0.1% Tween 20, and 5% skimmed milk) with shaking at room temperature for one hour. Thus, the membrane was blocked. After blocking, the membrane was washed with TBS-T (20 mM Tris (pH 7.6), 137 mM NaCl, and 0.1% Tween 20) for two minutes. Next, the membrane was immersed in a solution of a primary antibody (a 1:500 dilution of the primary antibody (anti-SELP8K antibody or anti-His-tag antibody (Rockland Immunochemicals Inc.) in TBS-T), and left to stand at 4° C. overnight for antibody reaction. After the reaction, the membrane was washed for five minutes in TBS-T four times and immersed in a solution of a secondary antibody capable of binding to the primary antibody and containing horseradish peroxidase as a marker enzyme (the solution of the secondary antibody was a 1:2000 dilution of the secondary antibody (ECL anti-rabbit IgG HRP linked F(ab′)2 fragment (GE Healthcare)) in TBS-T), and left to stand at room temperature for 30 minutes for antibody reaction. After the reaction, the membrane was washed for five minutes in TBS-T four times, and enzyme reaction was carried out using an ECL-Advance Western Blotting Detection kit (GE Healthcare). A luminometer ForECL (GE Healthcare) was used to expose the membrane to a high speed black and white instant film (Fujifilm Corporation) to visualize bands.

### Determination of Total Percentage of β-Turns and Random Coils

The total percentage of β-turns and random coils in the protein (A) in Production Example A1 was determined as described below.

### Determination of Total Percentage of β-Turns and Random Coils

The protein (A) in Production Example A1 was dissolved in deionized water (4° C.) to a concentration of 0.3 mg/mL to prepare an aqueous solution of the protein (A) in Production Example A1. The aqueous solution of the protein (A) in Production Example A1 was analyzed at a measurement temperature of 4° C. with a circular dichroism spectrometer (JASCO Corporation: J-820), and a total percentage of β-turns and random coils of the protein (A) was calculated using a secondary structure analysis program (JWSSE model: JASCO Corporation). Table 2 shows the results.

TABLE 2

| | | | | Ratio of total number of | Ratio of number | |
|---|---|---|---|---|---|---|
| | | | | amino acid residues in | of GAGAGS | |
| | | | Total | amino acid sequence (X) | sequence (2) to | |
| | | Amino acid | percentage | and amino acid sequence | total number of | |
| | | sequence of | (%) of β- | (X′) to the number of all | amino acid sequence | |
| | | protein (A) or | turns and | amino acid residues in | (X) and amino acid | |
| | | protein (A′) | random coils | protein (A) | sequence (X′) | Hydrophobicity |
| Production | A1 | Sequence of | 70.9 | 58.8% | 1:2 | 0.62 |
| Example | | SEQ ID No: 16 | | | | |
| | A2 | Sequence of | 66.8 | 58.8% | 1:2 | 0.62 |
| | | SEQ ID No: 16 | | | | |
| | A3 | Sequence of | 84.1 | 58.8% | 1:2 | 0.62 |
| | | SEQ ID No: 16 | | | | |
| | A4 | Sequence of | 61.2 | 58.8% | 1:2 | 0.62 |
| | | SEQ ID No: 16 | | | | |
| | A5 | Sequence of | 60.2 | 51.9% | 1:2 | 0.42 |
| | | SEQ ID No: 29 | | | | |
| Comparative | A1 | Sequence of | 58.1 | 58.8% | 1:2 | 0.62 |
| Production | | SEQ ID No: 16 | | | | |
| Example | A2 | Sequence of | 25.5 | 58.8% | 1:2 | 0.62 |
| | | SEQ ID No: 16 | | | | |
| | A3 | Sequence of | 85.6 | 58.8% | 1:2 | 0.62 |
| | | SEQ ID No: 16 | | | | |
| | A4 | Sequence of | 15.4 | 81.3% | — | 1.20 |
| | | SEQ ID No: 27 | | | | |
| | A5 | Sequence of | 62.4 | 20.9% | 3:1 | 0.41 |
| | | SEQ ID No: 28 | | | | |

### Production Example A2

A protein (A) of Production Example A2 was produced as in Production Example A1, except that a process of “5-2 Refolding (high dilution method)” described below was performed between the processes of “5: Anion exchange chromatography” and “6: Ultrafiltration” in “Purification of SELP8K (A11-2-1 (i))” in “Preparation of SELP8K (A11-2-1 (i)).” Then, the total percentage of β-turns and random coils was determined. Table 2 shows the result.

### 5-2: Refolding (High Dilution Method)

The eluted fraction from the anion exchange chromatography was mixed with a 10 M urea solution as a protein denaturant to give a 6 M urea solution. The solution was allowed to stand for 12 hours at 4° C. The resulting solution was transferred to a dialysis membrane (Viskase Companies, Inc.) and dialyzed for 12 hours against deionized water in an amount 10 times the volume of the eluted fraction. Subsequently, the deionized water was discarded and replaced with fresh deionized water in an amount 10 times the volume of the eluted fraction to dialyze for another 12 hours. After repeating this operation three more times, i.e., five times in total, the solution in the dialysis membrane was recovered.

### Production Example A3

A protein (A) of Production Example A3 was produced as in Production Example Al, except that a process of “5-3 Refolding (high dilution method)” described below was performed between the processes of “5: Anion exchange chromatography” and “6: Ultrafiltration” in “Purification of SELP8K (A11-2-1 (i))” in “Preparation of SELP8K (A11-2-1 (i)).” Then, the total percentage of β-turns and random coils was determined. Table 2 shows the result.

### 5-3: Refolding (High Dilution Method)

The eluted fraction from the anion exchange chromatography was mixed with a 10 M urea solution as a protein denaturant to give a 6 M urea solution. The solution was allowed to stand for 12 hours at 4° C. The resulting solution was transferred to a dialysis membrane (Viskase Companies, Inc.) and dialyzed for 12 hours against deionized water in an amount 10 times the volume of the eluted fraction. Subsequently, the deionized water was discarded and replaced with fresh deionized water in an amount three times the volume of the eluted fraction to dialyze for another 12 hours. After repeating the dialysis of the eluted fraction against deionized water in an amount of three times the volume of the eluted fraction for five more times, the solution in the dialysis membrane was recovered.

### Production Example A4

A protein (A) of Production Example A4 was produced as in Production Example A1, except that a process of “5′: Affinity chromatography” described below was performed instead of the process of “5: Anion exchange chromatography” in “Purification of SELP8K (A11-2-1 (i))” in “Preparation of SELP8K (A11-2-1 (i)).” Then, the total percentage of β-turns and random coils was determined. Table 2 shows the result.

### 5′: Affinity Chromatography

The protein separated by “4: Ultrafiltration” was purified by affinity chromatography (GE Healthcare, Ni Sepharose 6 Fast Flow) using a His-tag, thereby recovering an eluted fraction.

### Production Example A5

A protein (A) of Production Example A5 having a sequence of (29) having a molecular mass of about 30 kDa was produced as in Production Example A1, except that a “plasmid pPT0345-4 encoding SLP8K4” was used instead of the “plasmid pPT0345 encoding SELP8K.” Then, the total percentage of β-turns and random coils was determined. Table 2 shows the results.

### Comparative Production Example A1

A protein (A′) of Comparative Production Example A1 was produced as in Production Example A1, except that a process of “5′' : Affinity chromatography” described below was performed instead of the process of “5: Anion exchange chromatography” in “Purification of SELP8K (A11-2-1 (i)” in “Preparation of SELP8K (A11-2-1 (i)).” Then, the total percentage of β-turns and random coils was determined. Table 2 shows the result.

### 5″: Affinity Chromatography

The protein separated by “4: Ultrafiltration” was purified by affinity chromatography (Clontech, TALON® Single Step Columns) using a His-tag, thereby recovering an eluted fraction.

### Comparative Production Example A2

A protein (A′) of Comparative Production Example A2 was produced as in Production Example A1, except that the process of “5′: Affinity chromatography” was performed without performing the processes of “3: Ammonium sulfate precipitation,” “4: Ultrafiltration” and “5: Anion exchange chromatography” in “Purification of SELP8K (A11-2-1 (i))” in “Preparation of SELP8K (A11-2-1 (i)).” Then, the total percentage of β-turns and random coils was determined. Table 2 shows the result.

### Comparative Production Example A3

A protein (A′) of Comparative Production Example A3 was produced as in Production Example A1, except that the process of “5: Anion exchange chromatography” was not performed in “Purification of SELP8K (A11-2-1 (i))” in “Preparation of SELP8K (A11-2-1 (i)).” Then, the total percentage of β-turns and random coils was determined. Table 2 shows the result.

### Comparative Production Example A4

A protein (A′) of Comparative Production Example A4 having an amino acid sequence (27) set forth in SEQ ID No: 27 having a molecular mass of about 37 kDa was produced as in Production Example A1, except that a “plasmid pPT0102-2 encoding ELP1.2” was used instead of the “plasmid pPT0345 encoding SELP8K.” Then, the total percentage of β-turns and random coils was determined. Table 2 shows the result.

### Comparative Production Example A5

A protein (A′) of Comparative Production Example A5 having an amino acid sequence (28) set forth in SEQ ID No: 28 having a molecular mass of about 93 kDa was produced as in Production Example A1, except that a “plasmid pSY1398-1 encoding SLP4.1” was used instead of the “plasmid pPT0345 encoding SELPSK.” Then, the total percentage of β-turns and random coils was determined. Table 2 shows the result.

### Production Example B1: Production of Meniscus Tissue Fragment (B)

A meniscus tissue from a Japanese white rabbit (weight: 3.0 kg) was immobilized on a stage which was preliminarily sterilized in an autoclave.

The immobilized meniscus tissue was cut with a scalpel blade holder provided with scalpel blades arranged in a 200 μm grid pattern. Subsequently, the cut pieces were again cut with the scalpel blade holder into about 200-μm cubical pieces, thereby obtaining meniscus tissue fragments (B) of Production Example B1.

Randomly selected 100 fragments of the meniscus tissue fragments (B) of Production Example B1 were observed with an optical microscope to measure the lengths of the sides of the fragments, and a volume per fragment was calculated. The meniscus tissue fragments (B) of Production Example B1 were found to have a number average volume per fragment of 0.008 mm³.

### Production Example B2

Meniscus tissue fragments (B) of Production Example B2, which were cut into about 1,000-μm cubical pieces, were obtained as in Production Example B1, except that the distance between the scalpel blades was changed from 200 μm to 1,000 μm.

Randomly selected 100 fragments of the meniscus tissue fragments (B) of Production Example B2 were observed with an optical microscope to measure the lengths of the sides of the fragments, and a volume per fragment was calculated. The meniscus tissue fragments (B) of Production Example B2 were found to have a number average volume per fragment of 1.141 mm³.

### Production Example B3

Meniscus tissue fragments (B) of Production Example B3, which were finely cut into about 9,900-μm cubical pieces, were obtained as in Production Example B1, except that the distance between the scalpel blades was changed from 200 μm to 9,900 μm.

Randomly selected 100 fragments of the meniscus tissue fragments (B) of Production Example B3 were observed with an optical microscope to measure the lengths of the sides of the fragments, and a volume per fragment was calculated. The meniscus tissue fragments (B) of Production Example B3 were found to have a number average volume per fragment of 970 mm³.

### Production Example B4

Meniscus tissue fragments (B) of Production Example B4, which were finely cut into about 90-μm cubical pieces, were obtained as in Production Example B1, except that the distance between the scalpel blades was changed from 200 μm to 90 μm.

Randomly selected 100 fragments of the meniscus tissue fragments (B) of Production Example B4 were observed with an optical microscope to measure the lengths of the sides of the fragments, and a volume per fragment was calculated. The meniscus tissue fragments (B) of Production Example B4 were found to have a number average volume per fragment of 0.0007 mm³.

### Production Example B5

Meniscus tissue fragments (B) of Production Example B5, which were finely cut into about 10, 500-μm cubical pieces, were obtained as in Production Example B1, except that the distance between the scalpel blades was changed from 200 μm to 10,500 μm.

Randomly selected 100 fragments of the meniscus tissue fragments (B) of Production Example B5 were observed with an optical microscope to measure the lengths of the sides of the fragments, and a volume per fragment was calculated. The meniscus tissue fragments (B) of Production Example B5 were found to have a number average volume per fragment of 1,152 mm³.

### Examples 1 to 18 and Comparative Examples 1 to 5: Production of Meniscus Regeneration Material and Comparative Meniscus Regeneration Material

The protein of each production example or comparative production example and phosphate buffered saline (hereinafter, also simply referred to as PBS, pH 7.2) in amounts (parts by weight) indicated in Table 3 were mixed so that the protein was dissolved in the PBS. The solution and the meniscus tissue fragments of each production example in amounts (parts by weight) indicated in Table 3 were mixed by inversion, thereby producing a meniscus regeneration material of each example or comparative example.

### Meniscus Regeneration Test Using Meniscus Regeneration Material

The meniscus regeneration material of each example or comparative example was applied to a rabbit meniscus damage model.

Specifically, a knee of an anesthetized Japanese white rabbit (weight: 3.0 kg) was cut open, and a 2 mm-diameter round pillar-shaped defect was formed at an anterior corner of the meniscus.

The meniscus regeneration material was administered to the defect. The cut knee was sutured with a suture.

Four weeks later, the rabbit under sufficiently deep anesthesia was placed in a container filled with carbon dioxide, and cardiopulmonary arrest was confirmed. Next, a tissue including the defect was taken out. The tissue was immersed in 10% buffer formalin to be immobilized with formalin.

The tissue was embedded with paraffin and repeatedly sliced using a microtome (product name: RETORATOME REM-710, Yamato Kohki Industrial Co., Ltd.) in a direction perpendicular to the circular face of the cylindrical defect. Each slice obtained had a cross section having a thickness of about 4 μm.

One of the slices including the center of the circular face of the cylindrical defect was selected as a specimen for evaluations. The specimen was stained with hematoxylin-eosin (Evaluation items 1 and 2) or safranin O (Evaluation item 3). The evaluations were performed by the following methods using a tissue image captured by a digital microscope (VHX-2000, Keyence Corporation).

### Evaluation Item 1: Bound Repaired Tissues

The stained defect was observed with an optical microscope for evaluation based on the criteria described below. Three rabbits were tested for the evaluation. The value shown in Table 3 is an average of the scores.

A higher item score indicates higher meniscus regeneration ability.
2: Repaired tissues regenerated from both ends of the cylindrical defect in the cross section of the specimen, and the repaired tissues at the both ends were bound to each other.
1: Repaired tissues regenerated from both ends of the cylindrical defect in the cross section of the specimen, and the repaired tissues at the both ends were not bound to each other.
0: No repaired tissue regenerated from both ends of the cylindrical defect in the cross section of the specimen.

### Evaluation Item 2: Presence of Fibrocartilage Cells

The stained defect was observed with an optical microscope for evaluation based on the criteria described below. A higher item score indicates higher meniscus regeneration ability. Three rabbits were tested for the evaluation. The value shown in Table 3 is an average of the scores.
2: Fibrocartilage cells were widely present (the number of fibrocartilage cells per unit area: 2,000 cells/mm² or more) in the repaired tissues in the cross section of the specimen.
1: Fibrocartilage cells were partly present (the number of fibrocartilage cells per unit area: 100 cells/mm² or more but less than 2, 000 cells/mm²) in the repaired tissues in the cross section of the specimen.
0: No fibrocartilage cells were present (the number of fibrocartilage cells per unit area: less than 100 cells/mm²) in the repaired tissues in the cross section of the specimen.

The “number of fibrocartilage cells per unit area” was determined as follows: The defect of the specimen was observed with a fluorescent microscope (BIOREVO BZ-9000, Keyence Corporation) to count the number of fiber chondrocytes present in a 0.5 mm-square area. The counting was performed at five points including the center and four corners of the defect. The average value was determined as the “number of fibrocartilage cells per unit area.”

### Evaluation Item 3: Safranin O Staining

The stained defect was observed with an optical microscope for evaluation based on the criteria described below. A higher item score means a larger cartilage substrate production from the fibrocartilage cells and indicates higher meniscus regeneration ability. Three rabbits were tested for the evaluation. The value shown in Table 3 is an average of the scores.
2: The cross section of the specimen was densely stained (staining intensity: 65% or higher).
1: The cross section of the specimen was faintly stained (staining intensity: 33% or higher but lower than 65%).
0: The cross section was not stained (staining intensity: lower than 33% r).

The “staining intensity” was determined as follows: The defect of the specimen was observed with a fluorescent microscope (BIOREVO BZ-9000, Keyence Corporation). The Red/Green/Blue brightness distribution in a 0.5 mm-square area was obtained by histogram analysis. Using the data with a brightness of 150 or higher in the distribution, the percentage (%) of the brightness of red in the sum of the brightnesses of red, green, and blue was calculated (brightness of less than 150 was regarded as brightness 0). The ratio was determined at five points including the center and four corners of the defect. The average value was determined as the “staining intensity.”

TABLE 3

| | | Protein (A) or Protein (A′) | | PBS | Meniscus tissue fragment (B) | | | |
|---|---|---|---|---|---|---|---|---|
| | | | Parts | Parts | | Parts | Weight (%) of | Weight % of |
| | | | (parts by | (parts by | | (parts by | protein (A) or | meniscus tissue |
| | | Type | weight) | weight) | Type | weight) | protein (A′) | fragment (B) |
| Example | 1 | Production Example A1 | 12.5 | 62.50 | Production Example B1 | 25.0 | 12.5 | 25.0 |
| | 2 | Production Example A2 | 12.5 | 62.50 | Production Example B1 | 25.0 | 12.5 | 25.0 |
| | 3 | Production Example A3 | 12.5 | 62.50 | Production Example B1 | 25.0 | 12.5 | 25.0 |
| | 4 | Production Example A4 | 12.5 | 62.50 | Production Example B1 | 25.0 | 12.5 | 25.0 |
| | 5 | Production Example A5 | 12.5 | 62.50 | Production Example B1 | 25.0 | 12.5 | 25.0 |
| | 6 | Production Example A1 | 12.5 | 62.50 | Production Example B2 | 25.0 | 12.5 | 25.0 |
| | 7 | Production Example A1 | 12.5 | 62.50 | Production Example B3 | 25.0 | 12.5 | 25.0 |
| | 8 | Production Example A1 | 25.0 | 50.00 | Production Example B1 | 25.0 | 25.0 | 25.0 |
| | 9 | Production Example A1 | 12.5 | 37.50 | Production Example B1 | 50.0 | 12.5 | 50.0 |
| | 10 | Production Example A1 | 5.0 | 70.00 | Production Example B1 | 25.0 | 5.0 | 25.0 |
| | 11 | Production Example A1 | 25.0 | 70.00 | Production Example B1 | 5.0 | 25.0 | 5.0 |
| | 12 | Production Example A1 | 12.5 | 62.50 | Production Example B4 | 25.0 | 12.5 | 25.0 |
| | 13 | Production Example A1 | 12.5 | 62.50 | Production Example B5 | 25.0 | 12.5 | 25.0 |
| | 14 | Production Example A1 | 30.0 | 45.00 | Production Example B1 | 25.0 | 30.0 | 25.0 |
| | 15 | Production Example A1 | 12.5 | 35.00 | Production Example B1 | 52.5 | 12.5 | 52.5 |
| | 16 | Production Example A1 | 4.0 | 71.00 | Production Example B1 | 25.0 | 4.0 | 25.0 |
| | 17 | Production Example A1 | 12.5 | 83.75 | Production Example B1 | 3.8 | 12.5 | 3.8 |
| | 18 | Production Example A1 | 6.3 | 93.75 | — | 0.0 | 6.3 | 0.0 |
| Comparative | 1 | Comparative Production | 12.5 | 62.50 | Production Example B1 | 25.0 | 12.5 | 25.0 |
| Example | | Example A1 | | | | | | |
| | 2 | Comparative Production | 12.5 | 62.50 | Production Example B1 | 25.0 | 12.5 | 25.0 |
| | | Example A2 | | | | | | |
| | 3 | Comparative Production | 12.5 | 62.50 | Production Example B1 | 25.0 | 12.5 | 25.0 |
| | | Example A3 | | | | | | |
| | 4 | Comparative Production | 12.5 | 62.50 | Production Example B1 | 25.0 | 12.5 | 25.0 |
| | | Example A4 | | | | | | |
| | 5 | Comparative Production | 12.5 | 62.50 | Production Example B1 | 25.0 | 12.5 | 25.0 |
| | | Example A5 | | | | | | |

| | | Meniscus regeneration test | | | | | | | | |
|---|---|---|---|---|---|---|---|---|---|---|
| | | | Evaluation item 2 | | | | Evaluation item 3 | | | |
| | | Eval- | | Measured | Measured | Measured | | Measured | Measured | Measured |
| | | uation | | value of | value of | value of | | value of | value of | value of |
| | | item 1 | | 1st sample | 2nd sample | 3rd sample | | 1st sample | 2nd sample | 3rd sample |
| | | Score | Score | (cell/mm²) | (cell/mm²) | (cell/mm²) | Score | (%) | (%) | (%) |
| Example | 1 | 2.0 | 2.0 | 2482 | 2507 | 2461 | 2.0 | 86.8 | 84.7 | 79.5 |
| | 2 | 1.7 | 2.0 | 2398 | 2215 | 2257 | 2.0 | 88.2 | 77.9 | 79.9 |
| | 3 | 2.0 | 2.0 | 2496 | 2372 | 2252 | 2.0 | 85.3 | 84.7 | 78.0 |
| | 4 | 2.0 | 2.0 | 2426 | 2358 | 2206 | 2.0 | 88.4 | 88.3 | 77.3 |
| | 5 | 2.0 | 1.7 | 2097 | 2058 | 1955 | 2.0 | 73.1 | 76.4 | 69.5 |
| | 6 | 2.0 | 2.0 | 2494 | 2510 | 2396 | 1.7 | 81.7 | 75.8 | 64.5 |
| | 7 | 2.0 | 2.0 | 2545 | 2495 | 2310 | 1.7 | 80.8 | 75.2 | 64.0 |
| | 8 | 1.7 | 2.0 | 2357 | 2107 | 2006 | 1.7 | 74.1 | 75.1 | 61.8 |
| | 9 | 1.7 | 2.0 | 2328 | 2234 | 2138 | 1.7 | 70.3 | 75.1 | 63.6 |
| | 10 | 1.7 | 1.7 | 2053 | 2018 | 1869 | 2.0 | 84.8 | 72.6 | 70.6 |
| | 11 | 1.7 | 2.0 | 2107 | 2178 | 2245 | 2.0 | 78.2 | 80.2 | 86.6 |
| | 12 | 1.3 | 1.7 | 2027 | 2050 | 1803 | 1.7 | 70.1 | 73.3 | 58.6 |
| | 13 | 1.3 | 1.7 | 2030 | 2002 | 1748 | 1.3 | 65.2 | 55.2 | 47.6 |
| | 14 | 1.3 | 1.3 | 2020 | 1804 | 1706 | 1.3 | 65.3 | 50.3 | 50.4 |
| | 15 | 1.3 | 1.3 | 2008 | 1801 | 1701 | 1.3 | 66.7 | 50.1 | 50.2 |
| | 16 | 1.7 | 2.0 | 2108 | 2023 | 2019 | 1.3 | 65.4 | 51.2 | 55.4 |
| | 17 | 1.3 | 1.7 | 2007 | 2043 | 1840 | 1.3 | 67.2 | 53.3 | 50.5 |
| | 18 | 1.7 | 1.7 | 2003 | 2003 | 1914 | 1.3 | 65.5 | 59.1 | 50.4 |
| Comparative | 1 | 0.7 | 0.3 | 145 | 82 | 43 | 0.3 | 38.1 | 30.1 | 30.8 |
| Example | 2 | 0.7 | 0.3 | 132 | 73 | 45 | 0.3 | 35.5 | 26.8 | 28.7 |
| | 3 | 0.7 | 0.3 | 166 | 81 | 33 | 0.3 | 39.4 | 21.8 | 24.8 |
| | 4 | 0.3 | 0.3 | 157 | 80 | 33 | 0.3 | 36.2 | 26.2 | 26.6 |
| | 5 | 0.3 | 0.7 | 120 | 123 | 87 | 0.0 | 15.0 | 21.5 | 6.5 |

The evaluation results demonstrate that, in the cases of Examples, regeneration with repaired tissues occurred in the defect, and fibrocartilage cells were widely present in the repaired tissues. These Examples also exhibited good safranin O staining, which occurred at cartilage substrate presumably produced by the fibrocartilage cells.

### INDUSTRIAL APPLICABILITY

The meniscus regeneration material of the present invention can promote meniscus regeneration when used to fill a defect of the meniscus of the knee joint and can produce high quality cartilage tissues in the regeneration therapy of damaged meniscus. It is an effective substrate for meniscus regeneration.

1. A method of treating a damaged part of a meniscus, the method comprising;
a step applying a meniscus regeneration material to a damaged part of an exposed meniscus,
wherein the meniscus regeneration material comprises a protein (A),
the protein (A) consists of the amino acid sequence as set forth in SEQ ID No: 16 or SEQ ID No: 29, and
the protein (A) has a total percentage of β-turns and random coils of 60 to 85% as determined by circular dichroism spectroscopy.

2. The method of treating a damaged part of a meniscus according to claim 1,
wherein the meniscus regeneration material further comprises a meniscus tissue fragment (B).

3. The method of treating a damaged part of a meniscus according to claim 2,
wherein a number average volume per fragment of the meniscus tissue fragment (B) is 0.001 to 1,000 mm³.

4. The method of treating a damaged part of a meniscus according to claim 1,
wherein the meniscus regeneration material is in at least one form selected from the group consisting of solid, aqueous solution, and gel.

5. The method for treating a damaged part of a meniscus according to claim 4,
wherein the meniscus regeneration material is in the solid form, being at least one form selected from powder, sponge, fiber, sheet and non-woven fabric.

6. The method of treating a damaged part of a meniscus according to claim 1,
wherein the method further comprises a step gelling the meniscus regeneration material, and
wherein the meniscus regeneration material further comprises water.

7. The method of treating a damaged part of a meniscus according to claim 1,
wherein the method further comprises a step exposing the damaged part of the meniscus by incision.

8. A method of treating a damaged part of the meniscus, the method comprising;
a step applying the meniscus regeneration material to a damaged part of the meniscus under an arthroscope,
wherein the meniscus regeneration material comprises a protein (A),
the protein (A) consists of the amino acid sequence as set forth in SEQ ID No: 16 or SEQ ID No: 29, and
the protein (A) has a total percentage of B-turns and random coils of 60 to 85% as determined by circular dichroism spectroscopy.

9. The method of treating a damaged part of the meniscus according to claim 8,
wherein the meniscus regeneration material further comprises a meniscus tissue fragment (B).

10. The method of treating a damaged part of the meniscus according to claim 9,
wherein a number average volume per fragment of the meniscus tissue fragment (B) is 0.001 to 1,000 mm³.

11. The method of treating a damaged part of a meniscus according to claim 8,
wherein the meniscus regeneration material is in at least one form selected from the group consisting of solid, aqueous solution, and gel.

12. The method of treating a damaged part of a meniscus according to claim 11,
wherein the meniscus regeneration material is in the solid form, being at least one form selected from powder, sponge, fiber, sheet and non-woven fabric.

13. The method of treating a damaged part of a meniscus according to claim 8,
wherein the method further comprises a step gelling the meniscus regeneration material, and
wherein the meniscus regeneration material further comprises water.
